(19) United States
(12) Patent Application Publication
(10) Pub. No.: US 2025/0143648 A1
(43) Pub. Date: May 8, 2025

(54) System for Classifying Post Induction Blood Pressure Instability with an Automated Model

(71) Applicant: Becton, Dickinson and Company, Franklin Lakes, NJ (US)

(72) Inventors: Denise P. Veelo, Abcoude (NL); Alexander P.J. Vlaar, Bussum (NL); Jimmy Schenk, Amsterdam (NL); Eline Kho, Amsterdam (NL); Rogier V. Immink, Amersfoort (NL); Bjorn J.P. van der Ster, Rotterdam (NL)

(21) Appl. No.: 18/938,987

(22) Filed: Nov. 6, 2024

Related U.S. Application Data
(60) Provisional application No. 63/596,593, filed on Nov. 6, 2023.

(51) Int. Cl. A61B 5/00; A61B 5/021; G16H 10/60; G16H 50/70

(52) U.S. Cl. CPC A61B 5/7267; A61B 5/021; A61B 5/4848; A61B 5/7225; A61B 5/742; A61B 5/746; G16H 10/60; G16H 50/70

(57) ABSTRACT

Post induction hypotension (PIH) may be associated with an increase of morbidity and mortality. As PIH is caused by different (pharmacological) mechanisms compared to intra operative hypotension (IOH), using any of the various definitions for IOH is inadequate. Accordingly, the present application describes a more comprehensive method to assess clinically relevant PIH (defined as “crasher”) based on visual blood pressure patterns and an automated classification model able to classify these types of patients.

### CROSS REFERENCE

This application claims the benefit of U.S. Provisional Application No. 63/596,593 entitled Hemodynamic Sensor Systems For Defining Post Induction Blood Pressure Instability With An Automated Classification Model and filed on Nov. 6, 2023, the entirety of which is hereby incorporated by reference.

### FIELD

The present disclosure relates generally to hemodynamic monitoring, including prediction of post induction hypotension in a patient (e.g., human or veterinary subject) using monitored hemodynamic data.

### BACKGROUND

Monitoring hemodynamic variables of a patient allows for improved patient care. The hemodynamic variables can include heart health parameters, such as cardiac output. Monitoring such heart health parameters can allow a system to make diagnoses of an endotype of hypotension and provide interventions in hypotensive or potentially hypotensive patients. Systems and methods described herein provide potentially life-saving solutions in the space.

Post induction hypotension (PIH) may be associated with an increase of morbidity and mortality. Its incidence shows a wide distribution as it is affected by a chosen definition of hypotension, based on absolute or relative thresholds. In this disclosure a more comprehensive method to assess clinically relevant PIH, incorporating the magnitude and the speed of blood pressure changes, based on visual blood pressure patterns and aimed to develop an automated classification model, is developed to classify these types of patients.

### SUMMARY

Systems and methods for classifying a patient who experiences post-induction hypotension (PIH). The system can include a non-transitory memory that can stores sensed hemodynamic data representative of an arterial pressure waveform of the patient. The non-transitory memory can have executable instructions stored thereon including a PIH classification model. An electronic hardware processor in communication with the non-transitory memory and can be configured to execute the instruction to cause the system to at least: extract a plurality of signal measures from the arterial pressure waveform of the patient; extract input features from the plurality of signal measures; determine, based on the extracted input features, a PIH classification of the patient as a PIH crasher or PIH non-crasher using the PIH classification model; and generate, based on the determined PIH classification of the patient, data for displaying an alert indicating the determined PIH classification of the patient or otherwise recording the PIH classification.

### BRIEF DESCRIPTION

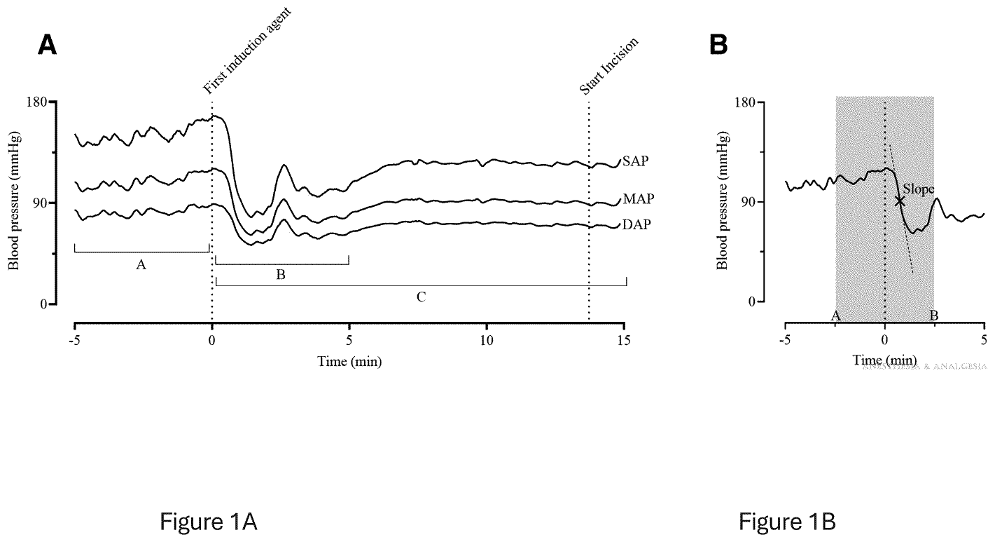

FIG. 1A shows a hemodynamic overview of a patient who crashes after induction of anesthesia showing the averaged systolic (SAP), mean (MAP) and diastolic blood pressure (DAP). Data were split into three sections: (A) a first time period of blood pressure data before the first induction agent; (B) a second time period of blood pressure data after the first induction agent; and (C) a third time period of blood pressure data after the first induction agent, which can be longer than and overlapping with the second time period. Multiple waveform features can be derived from each of these sections of data.

FIG. 1B shows a time period of the patient hemodynamics before and after induction (grey area) is implemented to derive a maximum slope value.

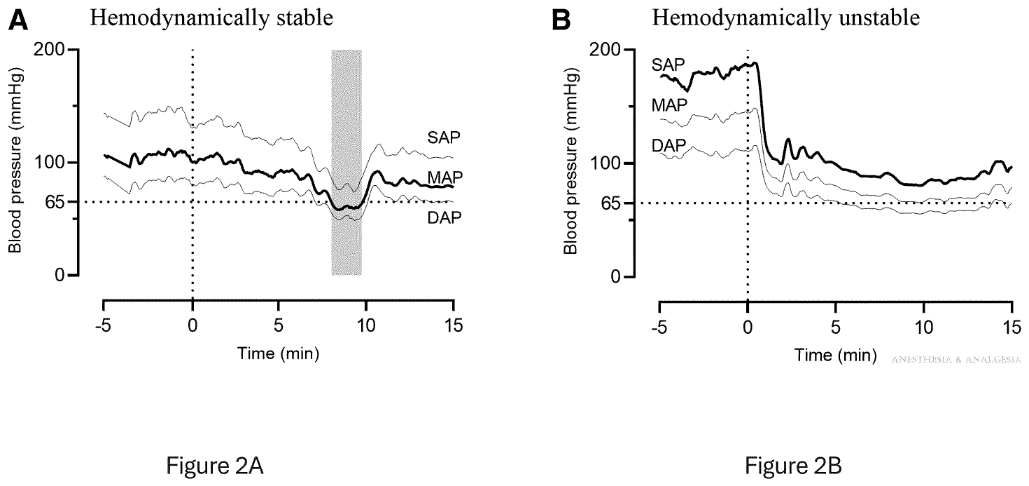

FIG. 2A shows a hemodynamic overview of a typical examples of mismatch between a common intraoperative hypotension definition (below 65 mmHg) and the PIH definition according to the present disclosure. The blood pressure tracing illustrated is not considered of clinical major interest, as blood pressure declines slowly. However, with the hypotension definition of MAP<65 mm Hg, this patient would be classified as hypotensive.

FIG. 2B is another example of mismatch between a common intraoperative hypotension definition (e.g., below 65 mmHg) and the PIH definition according to the present disclosure. Here the a hemodynamically unstable patient is illustrated; however, the patient would not have been classified as not having experience hypotension, when applying a typical the hypotension definition of MAP<65 mmHg or SAP<65 mmHg post-induction hypotension.

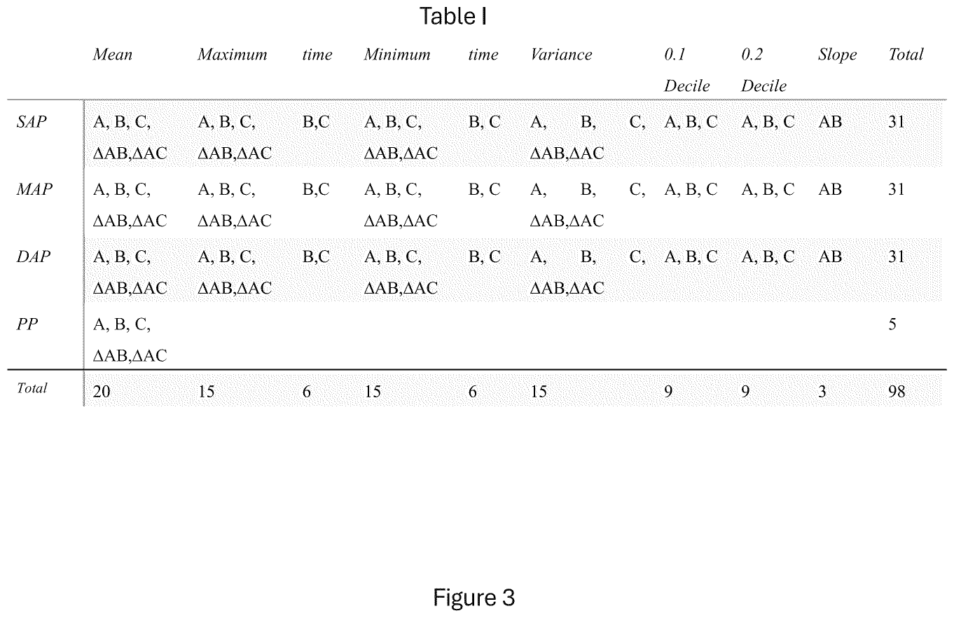

FIG. 3 shows Table I with an overview of blood pressure features implemented in one classification model. Features can be calculated from the systolic, mean, and diastolic blood pressure (SAP, MAP, DAP) and the pulse pressure (PP), in sections A, B, and C (FIG. 1A). Additionally, differences in the features between sections (AAB or AAC) can be calculated. The slope can be calculated in blood pressure data for a period spanning before and after the first induction agent.

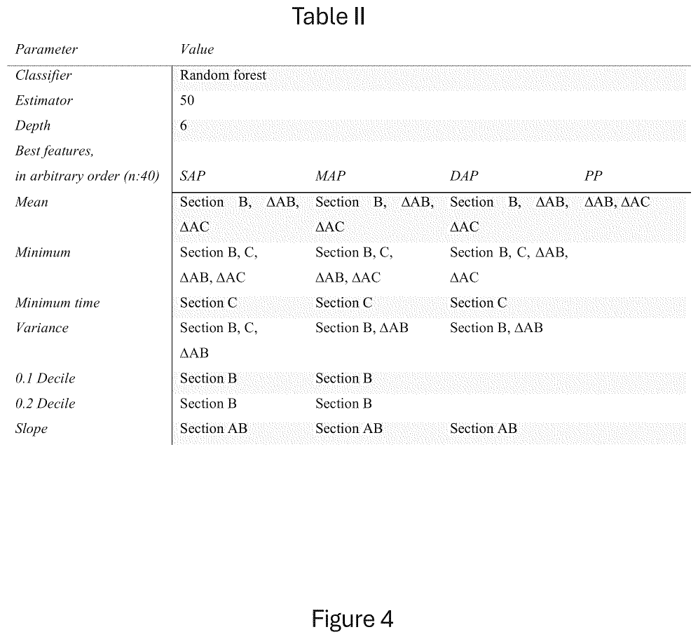

FIG. 4 shows Table II with exemplary parameters for one type of trained classification model (Random Forest).

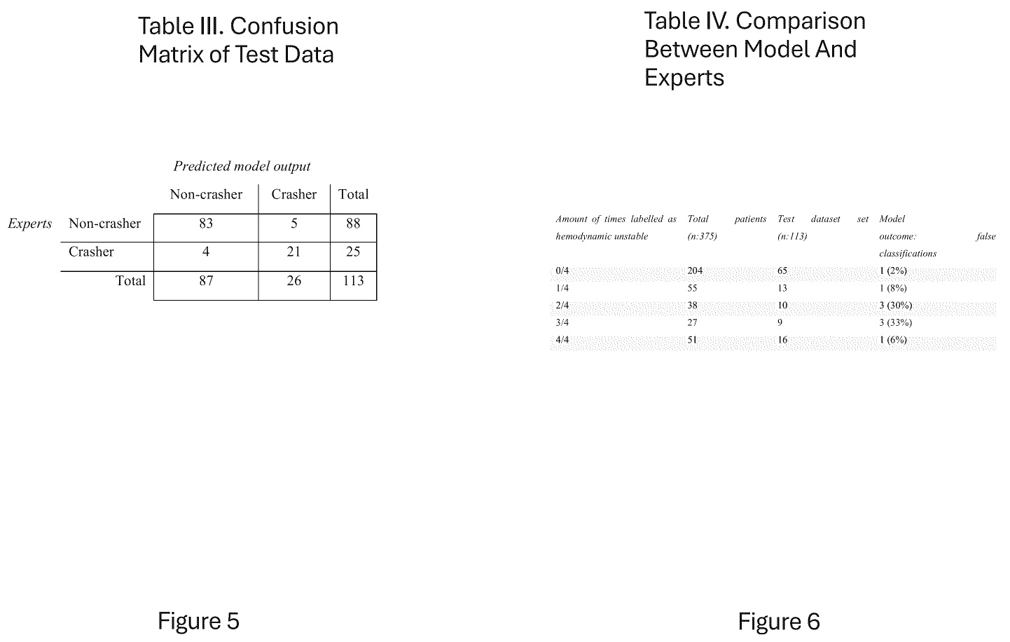

FIG. 5 shows Table III with a confusion matrix of test dataset.

FIG. 6 shows Table IV with a comparison of classification results from human experts and trained classification models according to the present disclosure.

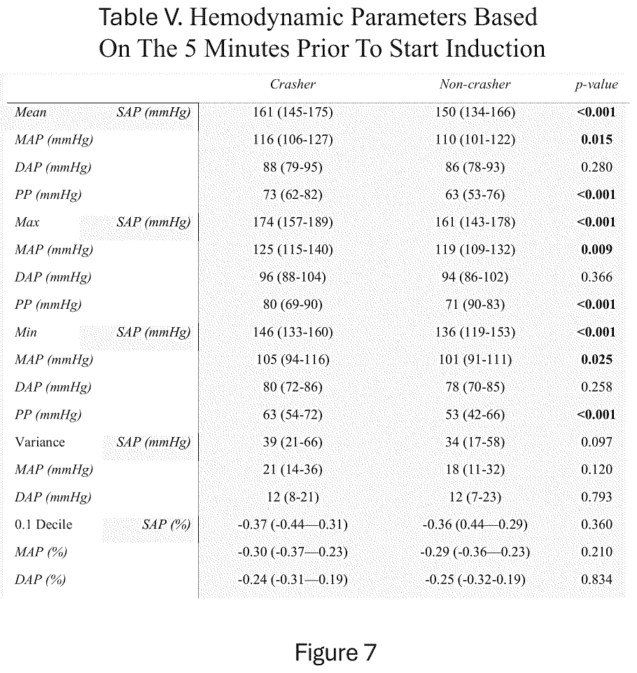

FIG. 7 shows Table V with a plurality of exemplary hemodynamic parameters prior to the start of induction.

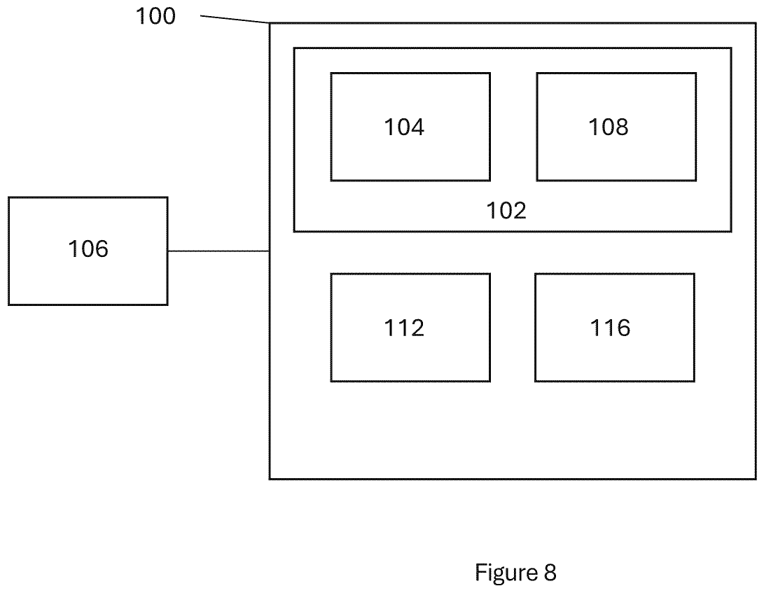

FIG. 8 shows a block diagram of a classification system for identifying patients who experience post-induction hypotension (crashers) and patients who are hemodynamically stable post-induction (non-crashers).

### DETAILED DESCRIPTION

### Overview

Hypotension can arise fast and intense during the induction of anesthesia with incidences ranging between 18 and 60%. It often occurs unexpectedly, and correction is initiated too late since the attention of anesthesiologists is deviated to other activities during this part of the intraoperative phase. The wide distribution of post induction hypotension (PIH) incidence is affected by the chosen definition of hypotension, based on absolute or relative thresholds. The higher the chosen threshold, the higher the incidence.

Describing hypotension solely based on an absolute or relative threshold seems illogical. First, to be classified as hypotensive, patients with a high blood pressure will have to endure a major drop in blood pressure when using an absolute threshold, but only requires a minor drop when a relative threshold is used. When a patient has a low awake blood pressure, the absolute decline in blood pressure to reach the hypotensive group is comparable to the relative threshold. Second, the speed at which blood pressure drops is rarely taken into consideration. Patients who merely drift towards a lower blood pressure can easily be intervened, while patients who show a crash in blood pressure cannot. This way, patients who rapidly drop in blood pressure but remain above a blood pressure threshold might not be classified as hypotensive, while physicians might feel the urge to intervene.

In this disclosure, a conversion towards a more comprehensive way to define this hemodynamic instability is proposed, to incorporate the magnitude and the speed of blood pressure changes. Therefore, a set of blood pressure tracings was presented to physicians who visually decided which patients hemodynamically crashed during induction and who did not. Based on their decisions, an automated classification model that can differentiate between patients who are stable and who are not was developed.

### Methods

To obtain relevant study date, beat-to-beat non-invasive blood pressure data can be obtained noninvasively. In certain examples, a blood-pressure finger cuff connected to the Nexfin device (BMEYE, Amsterdam, the Netherlands) or its successor, the HemoSphere monitor (Edwards Lifesciences, Irvine, CA, USA) can be used. Data from the radial finger blood pressure can be remodeled to a brachial blood pressure (BP). A description of this non-invasive BP measurement technique can be found elsewhere. One or more of these devices can contain the same algorithm that automatically derives beat-to-beat parameters, such as the systolic, mean, and diastolic arterial blood pressures (SAP, MAP, and DAP, respectively). Measurements started after arrival at the operating theatre and lasted until end of surgery. During measurements, the administration, timing, and dosing of medication was annotated by a trained researcher.

In one example, 20 minutes of BP data during induction can be extracted and/or used for determinations, starting 5 minutes before, and ending 15 minutes after onset of induction (defined as the moment of administration of the first induction agent). Patients may be excluded from analyses when more than a threshold amount of BP data is missing (>4 minutes within the 5 minutes before or after start induction, and/or >8 minutes in the last 10 minutes). The MAP, SAP, and DAP values may be eliminated in case of unrealistic values (a pulse pressure (PP)<10 mmHg, an SAP change >20 mmHg between consecutive beats, or when the difference between DAP and the ten enclosing DAP values was >25 mmHg), and/or when the heart reference system (HRS) is unaligned (change of >35 cm from start). The remaining beat-to-beat values may be interpolated to up to 1 Hz, which may be followed by a moving average of 45 seconds.

Multiple features may be extracted from the pre-processed data described above and/or used as input for the development of the hemodynamic instability detection model. The 20 minutes of induction may be divided into two or more (e.g., three) (partly overlapping) sections; the 5 minutes before induction, the 5 minutes after induction, and/or the 15 minutes after induction (Figure I, sections A, B and C, respectively). For each section the lowest and highest value (with corresponding timestamp), mean, and variance of SAP, MAP, and DAP may be determined. Besides those, PP and the mean negative slope, based on the BP 2.5 minutes before and after induction, Figure I (B), may be derived. The first and second decile of the steepest downwards slopes can be stored for SAP, MAP, and DAP. Change over time of the parameters between the 3 different sections (for example the change in mean SAP; comparing section A to section B) may be calculated. As shown in Table I, a total of 98 features were derived in some embodiments.

Patients who are deemed to have a decrease in BP that was of clinical interest (for example, a deep or rapid decrease in BP) during induction (e.g., severe hemodynamic instability) can be defined as crashers, having clinically relevant PIH. Patients with a normal or a clinical insignificant, slowly developing decrease in BP are defined as non-crashers/not having PIH. As referenced above, using a fixed threshold may lead to incorrect labelling of patients. For example, implementing a commonly used threshold of MAP<65 mmHg may result in the classification of a patient as having experienced hypotension, while the reduction in blood pressure may be actually very small (FIG. 2(A)). Contrarily, a patient starting off in hypertension can illustrate a major reduction in blood pressure but may not be labelled as having experienced hypotension (FIG. 2(B)).

In one embodiment, an expert panel (n=15) consisting of experienced physicians in this field, working either at the intensive care or anesthesiology department of the Amsterdam UMC, individually assigned whether a patient was a crasher or not. This was based on 2 Figures, containing the raw non-invasive blood pressure trend, and the 45 seconds averaged SAP, MAP, and DAP tracings. Exact information such as timings of induction, intubation, nasogastric positioning, administration of anesthetics, and vasopressors were annotated in the figures. Experts labelled patients as crashers when they considered the patients' hemodynamic status unstable and/or of clinical interest. No strict definition of a crasher was given at the time of labelling. In total 75 cases were labelled by all physicians, of which 25 cases were labelled twice, without their knowledge. When at least 75% of the expert panel agreed, the patient was classified as a crasher, and otherwise treated as a non-crasher.

Several reliability analyses were performed to assess the labelling of the experts. These analyses calculate a correlation coefficient, where values range between 0 and 1. A value between 0.50 and 0.75 indicates moderate reliability, 0.75 and 0.90 indicates good reliability, and a value above 0.90 represent excellent reliability.

First, the consistency of the experts was determined with the 25 doubly labelled patients. This was calculated with intra-rater reliability analyses, derived for each expert, and averaged, employing a two-way mixed effect model, with data treated as single measures. Second, the agreement, or inter-rater reliability, between the experts was determined with the two-way random and average measures. Third, the average inter-rater reliability of the labels of four experts were compared to that of the other eleven experts. This would allow us to determine whether four experts could accurately reflect the integral expert outcome, when labelling additional cases. For the inter-rater reliability analyses, the two-way random effect model, with average measures, was applied. In case of good consistency, defined as reliability ≥0.80, these four experts were deemed able to label all remaining patients.

The overall dataset was divided into a training and test set, consisting out of 70% and 30% of the samples, respectively. Both sets were normalized by scaling each feature between 0 and 1 and used to derive the best performing classifier and hyperparameters through a grid search. The tested classifiers algorithms were; logistic regression, K-nearest neighbors, support vector machine and random forest. The models were trained towards the area under the receiver operating curve (AUROC). To solve the imbalanced dataset, Synthetic Minority Over-sampling Technique (SMOTE) was applied on the training dataset.

The incidence of PIH found with the classification model was compared to the incidence of PIH when applying more commonly used PIH definitions. First, the incidence of PIH when using a MAP<65 mmHg as threshold was checked, and the second implemented definition was a decline in SAP>20% from baseline.

Differences in anesthetics and vasopressors between crashers and non-crashers were also investigated. As some anesthetics might result in a decrease in blood pressure, a vasopressor, like phenylephrine, ephedrine and noradrenalin, can be administered as a precaution. To distinguish vasopressors administered as a precaution from those to treat hypotension, a cut-off time was implemented, defined as the timing of the first rocuronium administration. In case rocuronium was not administered (when the patient was not intubated), this timing was based on the averaged time of the rocuronium administration found for the other patients.

Data was presented as mean with standard deviation (SD) or median (1st-3rd quartile), depending on the distribution. Differences between the hemodynamic unstable and other patients were assessed with either the unpaired t-test or the Wilcoxon rank sum test for continuous data, and with the Fischer's exact test for categorical data. Pre-processing and featurization was executed using MATLAB (Version 2020b, The Mathworks Inc., Nattick, MA, USA), reliability analyses with MedCalc (MedCalc Software, Ostend, Belgium), and the model development with the scikit-learn module in Python (Python Software Foundation. version 3.9, Scikit-learn 1.1.3).

### Results

In one embodiment, written informed consent was obtained from a total of 477 patients, of which 38 were excluded due to logistic problems, 7 due to withdrawal of consent, and 57 patients due to too much missing data. Of the remaining 375 patients, a median of 8.7% (5.8-13.4 1st-3rd quartile), of the SAP/MAP/DAP values were eliminated.

The data set (188 females vs 187 males) showed a good distribution in age and body mass index, with an average (SD) age of 57 (15) years, and body mass index of 26.6 (4.4) kg/m2. Most of the population has an ASA score of II (53%) or III (29%), and 32% of the patients had chronic hypertension.

In total 15 experienced physicians labelled the patients; seven with a background in intensive care medicine and eight with a background in anesthesiology. The intensive care physicians labelled 27% of the patients as crashers whereas the anesthesiologists labelled 31% as crashers. Intra-rater reliability of the 15 experts showed good agreement with a correlation of 0.80 (95% CI: 0.61-0.90). Concerning the inter-rater agreement, comparing experts with each other, an excellent agreement of 0.92 (95% CI: 0.89-0.94) was found. When comparing the average outcome of four experts with eleven experts for consistency, a correlation of 0.87 (95% CI: 0.80-0.92) was found, representing good agreement. Based on this, labelling by four experts was considered adequate and was applied on the remaining dataset.

In total 21% of the patients were classified as a crasher and 79% as non-crashers by the experts. When a PIH definition of MAP<65 mmHg was applied to the dataset, 26% of the patients were classified as hypotensive, of which 44% were also classified as crashers according to the new PIH approach. With a PIH definition of a decrease in SAP>20% from baseline, 47% of the patients were classified as hypotensive, of which 32% were also classified as crasher.

Based on the experts labelling, patients who crashed during induction were significantly older (with 7 years, p<0.001), and had a higher prevalence of COPD (10% vs 4%, p=0.036) compared to non-crashers. No significant differences were found in other patient characteristics such as height, weight, BMI, history, type of surgery, or preoperative medication. A higher pre-induction blood pressure was found in patients crashing. Here, SAP was increased (11 mmHg, p<0.001), consequently resulting in an increased PP and MAP (10 mmHg, p<0.001, and 6 mmHg, p=0.015).

Comparing the administered medication during the entire induction period, crashers received phenylephrine with a higher incidence (18% vs 9%, p=0.039) and a higher dosage (1.4 vs 1.3 μg·kg-1, p=0.028). Noradrenaline also was administered more often in crashers (77% vs 64%, p=0.039), with a higher dosage (0.47 vs 0.35 μg·kg-1·hr-1, p=0.007). Likewise, ephedrine boluses were administered more often in crashers than non-crasher (23% vs 8%, p<0.001). When administered, crashers received more remifentanil (6.8 vs 5.2 μg·kg-1·hr-1, p=0.042).

To examine whether the treatment/prevention of (early) hypotension had taken place, medication administered until the first rocuronium administration (with a mean average of 140 (94 SD) seconds after the first induction agent) was scrutinized. Here, crashers received more phenylephrine as compared to non-crashers (0.608 μg kg-1, p=0.042), and more crashers received noradrenaline perfusion (65 vs 44%, p=0.001).

The one exemplary model was the random forest classifier model, with 50 estimators and a maximum depth of 6 as the optimized parameters, Table II. Here, an AUROC of 0.98 (SD: 0.01) was found, based on the training set. Other classifiers showed similar results, with an AUROC (SD) of 0.98 (0.01) for K-nearest neighbors, 0.97 (0.02) for logistic regression and 0.95 (0.03) for the decision tree. Applying the random forest model to the test set resulted in an AUROC of 0.96, with a sensitivity of 0.84, specificity of 0.94, and an accuracy was 0.92. The test dataset consisted out of 25 crashers, and 88 non-crashers. Of those 88 patients, 5 patients were incorrectly identified as crashers, whereas 4 out of 25 crashers were incorrectly identified as non-crashers, Table III.

Herein is presented an automated classification model for post induction hypotension. This model is trained on features of the arterial blood pressure and assigned labels were based on visual differentiation by experts between patients with or without post induction hemodynamic instability. This way, clinically relevant (e.g., post induction) hypotension can be distinguished in an automated way from irrelevant hypotension or normotension.

Compared to commonly implemented PIH definitions using blood pressure thresholds, the incidence of PIH patients in one embodiment was reduced by more than half. In addition, different patients were identified as PIH. The labelled crashers tended to be older, with a higher incidence of COPD, and displayed higher blood pressures. The classification model showed excellent performance, making automated labelling of these patients feasible in large databases and pave the way for future research in the field of PIH.

In previous studies analyzing PIH, the thresholds for IOH, like an absolute value for MAP, a percentage decline in BP, or a combination of thresholds, are used. However, PIH is caused by different physical and pharmacological mechanisms compared to IOH. During induction of anesthesia, the instant administration of (bolus) anesthetics, such as propofol and opioids causes arterial and venous vasodilatation and reduces cardiac contractility leading to reductions in preload and afterload. Moreover, the heart rate is minimally affected as baroreflex sensitivity is depressed. Hence, applying static IOH definitions to assess PIH might be insufficient. In previous studies concerning PIH, its incidence ranges from 23% to 92%, making comparison between studies analyzing risk factors and outcomes challenging. By narrowing the definition for PIH, such as MAP<55 mmHg, the incidence will be lower. By contrast, for a broader definition, such as MAP<65 mmHg, the incidence will be higher. In some embodiments, the classification model may include multiple features from the blood pressure tracing to capture the hypotension of clinical interest. In one embodiment, the model is trained on labels based on visual interpretation of experts, the defined PIH entails a more physiological rationale. Distinguishing PIH patients, or crashers, from non-crashers with visual blood pressure patterns was feasible with good consensus between experts. The labelling was very strict, as experts were only eligible to label patients as crashers or non-crashers, while several patients were within the grey area between the labels. This can result in a lower intra-rater reliability of the experts, and a lower inter-rater reliability between experts concerning the Fleiss' kappa. Therefore, the Two-Way Random effect model to assess the inter-rater reliability was implemented, as this test is based on continuous values, partly omitting the two label restrictions. With the classification model, it was found that most of the patients labelled as crashers by all experts were correctly identified by the model. In some embodiments, the system may use external validation of experts of other hospitals. This model could aid in further research in predicting, and as a result preventing, PIH and possible related adverse outcomes.

In one embodiment, crashers showed an association with increased age, incidence of COPD, and a high SAP based on pre-induction blood pressures. These associations were also found in other studies, reporting associations with age, sex, weight, type 2 diabetes, MAP and SAP. Of interest is the reported inconsistency in the association of pre-induction MAP or SAP with the occurrence of PIH. One study showed that a MAP<70 mmHg was found positively related to PIH incidence, whereas other studies found a relation with an increased MAP. For SAP, a similar inconsistency was found, where both a lower and higher SAP were associated with PIH. Both studies included similar patients, and used a comparable pre-induction period to measure BP. These conflicting results may be explained using different definitions of hypotension, but also by the difference in premedication or the different stages of anxiety of the patients. Focusing on our current study, the higher blood pressure in the PIH group might be induced by the physician; it was found a higher incidence of noradrenaline administration in the PIH group. This might suggest that physicians anticipated these patients to become hemodynamic instable.

An automated classification model distinguishes between the presence and absence of clinically relevant PIH, based on blood pressure visualization. In creating this model, hemodynamic instable patients can now automatically be classified, contributing in further research concerning PIH, its risk factors, and its prevention.

FIG. 8 shows a block diagram for an automated classification system 100 for identifying post-induction hypotension crashers, as described above and further delineated below. The system 100 can include a non-transitory, computer-readable memory system 102. The memory system 102 can store, or be in communication with a remote storage system containing, sensed hemodynamic data 104 representative of an arterial pressure waveform of one or a plurality of patients. The hemodynamic data can include raw or processed waveform data (e.g., analog or digital).

The hemodynamic data 104 can be received from a hemodynamic monitoring sensor 106. The sensor 106 can comprise an arterial line blood pressure sensor (disposable pressure transducer) or a noninvasive sensor cuff such as the blood-pressure finger cuff described above. The system 100 can include an analog-to-digital converter that converts an analog hemodynamic sensor signal from the sensor 106 to the arterial pressure signal waveform of the hemodynamic data 104.

The system 100 can include a PIH classification model 108. The PIH classification model 108 can be stored on the memory system 102, or otherwise stored on a remote storage system in communication with the system 100.

The system 100 can include a processor containing instructions thereon to execute the PIH classification model 108 to analyze the hemodynamic data 104 and determine a PIH classification of each of the one or more datasets 102 as a crasher or non-crasher due to post-induction hypotension. The system 100 can include a user interface 116 for displaying an alert indicating the determined PIH classification of the patient or otherwise recording the PIH for future use.

The PIH classification model 108 can be trained using the techniques and methods described above. Training the PIH classification model 108 can include collecting a clinical dataset containing arterial pressure waveforms from a first group of individuals classified as PIH crashers and a second group of individuals classified as PIH non-crashers. Each of the individual arterial pressure waveforms can cover a time including an induction event. The induction event can divide each of the arterial pressure waveforms into a pre-induction event time period (like Section A in FIG. 1) and one or more post-induction event time periods (like Section B and C in Figure).

The clinical dataset can be mined to derive a plurality of waveform signal features. These features can include: systolic, mean, and diastolic blood pressure (SAP, MAP, DAP), the pulse pressure (PP), for both the pre-induction event time period and the post-induction event time period, or any of the features shown in Tables I or II.

The plurality of waveform signal features can be input into a classifier algorithm, such as the random forest classification model or any of the other classification models described. The results of the model can be iterated and optimized. In one example, the classifier algorithm can be optimized (e.g., maximized, minimized) towards the area under the receiver operating curve (AUROC) to derive the final PIH classification model 108. For example, the model may seek to reduce a number of classification errors. In certain examples, the plurality of waveform signal features can be normalized and the normalized features can be input into the classifier algorithm.

What is claimed is:

1. A system for generating an alert related to a classification of a patient experiencing post-induction hypotension (PIH), the system comprising:
a non-transitory memory that stores sensed hemodynamic data representative of an arterial pressure waveform of the patient;
the non-transitory memory having executable instructions stored thereon including a PIH classification model; and
an electronic hardware processor in communication with the non-transitory memory and configured to execute the instruction to cause the system to at least:
extract a plurality of signal measures from the arterial pressure waveform of the patient;
extract input features from the plurality of signal measures;
determine, based on the extracted input features, a PIH classification of the patient as a PIH crasher or PIH non-crasher using the PIH classification model; and
generate, based on the determined PIH classification of the patient, data for displaying an alert indicating the determined PIH classification of the patient.

2. The system of claim 1, wherein the PIH classification model is determined by machine training, the machine training comprising:
collecting a clinical dataset containing arterial pressure waveforms from a first group of individuals classified as PIH crashers and a second group of individuals classified as PIH non-crashers, each of an associated plurality of individual arterial pressure waveforms covering a time period including an induction event;
dividing each of the arterial pressure waveforms into a pre-induction event time period and a post-induction event time period;
performing waveform analysis of the arterial pressure waveforms for the pre-induction event time period and the post-induction event time period to calculate a plurality of waveform signal features;
inputting the plurality of waveform signal features into a classifier algorithm; and
increasing, using the classifier algorithm, an area under a receiver operating curve (AUROC) to derive the PIH classification model.

3. The system of claim 2, wherein the plurality of waveform signal features comprises: systolic, mean, and diastolic blood pressure (SAP, MAP, DAP) and a pulse pressure (PP), each for both the pre-induction event time period and the post-induction event time period.

4. The system of claim 3, wherein the post-induction event time period comprises a first post-induction event time period and a second post-induction event time period that is longer length than and overlapping with the first post-induction event time period, wherein the plurality of waveform signal features include differences in the waveform signal features between the pre-induction event time period and the first post-induction event time period and between the pre-induction event time period and the second post-induction event time period.

5. The system of claim 2, further comprising:
normalizing the plurality of waveform signal features; and
inputting the normalized plurality of waveform signal features into the classifier algorithm.

6. The system of claim 1 wherein the alert further indicates at least one of the extracted input features.

7. The system of claim 1, wherein the PIH classification model is one of: a logistic regression, a K-nearest neighbors, a support vector, a convoluted neural network, and a random forest classifier.

8. An method for automated classification system for patients who experience post-induction hypotension (PIH), the method comprising:
obtaining sensed hemodynamic data representative of an arterial pressure waveform of a patient;
extracting a plurality of signal measures from the arterial pressure waveform of the patient;
extracting input features from the plurality of signal measures;
determining, based on the extracted input features, a PIH classification of the patient as a PIH crasher or PIH non-crasher using a PIH classification model; and
generating, based on the determined PIH classification of the patient, data for displaying an alert indicating the determined PIH classification of the patient.

9. The method of claim 8, further comprising:
collecting a clinical dataset containing arterial pressure waveforms from a first group of individuals classified as PIH crashers and a second group of individuals classified as PIH non-crashers, each of an associated plurality of individual arterial pressure waveforms covering a time period including an induction event;
dividing each of the arterial pressure waveforms into a pre-induction event time period and a post-induction event time period;
performing waveform analysis of the arterial pressure waveforms for the pre-induction event time period and the post-induction event time period to calculate a plurality of waveform signal features;
inputting the plurality of waveform signal features into a classifier algorithm; and
increasing, using the classifier algorithm, an area under a receiver operating curve (AUROC) to derive the PIH classification model.

10. The method of claim 9, wherein the plurality of waveform signal features comprises: systolic, mean, and diastolic blood pressure (SAP, MAP, DAP) and a pulse pressure (PP), each for both the pre-induction event time period and the post-induction event time period.

11. The method of claim 10, wherein the post-induction event time period comprises a first post-induction event time period and a second post-induction event time period that is longer length than and overlapping with the first post-induction event time period, wherein the plurality of waveform signal features include differences in the waveform signal features between the pre-induction event time period and the first post-induction event time period and between the pre-induction event time period and the second post-induction event time period.

12. The method of claim 9, further comprising:
normalizing the plurality of waveform signal features; and
inputting the normalized plurality of waveform signal features into a classifier algorithm.

13. The method of claim 8, wherein the PIH classification model is one of: a logistic regression, a K-nearest neighbors, a support vector, a convoluted neural network, and a random forest classifier.

14. A hemodynamic sensor system for classifying patients based on a risk of post-induction hypotension (PIH), the system comprising:
a hemodynamic sensor that produces an analog hemodynamic sensor signal representative of an arterial pressure signal waveform of the patient;
an analog-to-digital converter that converts the analog hemodynamic sensor signal to the arterial pressure signal waveform;
a non-transitory memory having executable instructions stored thereon; and
an electronic hardware processor in communication with the non-transitory memory and configured to execute the instruction to cause the system to at least:
receive, from the hemodynamic sensor, the analog hemodynamic sensor signal from the patient;
convert, using the analog-to-digital converter, the analog hemodynamic sensor signal to the arterial pressure signal waveform;
extract a plurality of signal measures from the arterial pressure waveform of the patient;
extract input features from the plurality of signal measures;
determine, based on the extracted input features, a PIH classification of the patient as a PIH crasher or PIH non-crasher using a PIH classification model; and
generate, based on the determined PIH classification of the patient, data for displaying an alert indicating the determined PIH classification of the patient.

15. The system of claim 14, wherein the PIH classification model is determined by machine training, the machine training comprising:
collecting a first clinical dataset containing arterial pressure waveforms from a first group of individuals classified as PIH crashers;
collecting a second clinical dataset containing arterial pressure waveforms from a first group of individuals classified as PIH non-crashers;
performing waveform analysis of the arterial pressure waveforms of the first clinical dataset and the second clinical dataset to calculate a plurality of waveform signal features;
inputting the plurality of waveform signal features into a classifier algorithm; and
reducing a number of classification errors.

16. The system of claim 15, wherein the plurality of waveform signal features comprise: systolic, mean, and diastolic blood pressure (SAP, MAP, DAP) and a pulse pressure (PP), each for a first period before an induction time, a second period immediately after the induction time, and a third time period after the induction time having a longer length than and overlapping with the second time period, and differences in the waveform signal features between the first and second time periods and between the first and third time periods are calculated.

17. The system of claim 15, further comprising:
normalizing the waveform signal features; and
determining the input features based on training the PIH classification model on the normalized waveform signal features.

18. The system of claim 14 wherein the PIH classification model is a logistic regression, K-nearest neighbors, support vector machine, a convoluted neural network or random forest.

19. The system of claim 14 wherein the alert further indicates the at least one of the input features.
